(19) United States
(12) Patent Application Publication
(10) Pub. No.: US 2025/0090018 A1
(43) Pub. Date: Mar. 20, 2025

(54) PUPILLOMETER

(71) Applicants: The Children's Hospital of Philadelphia, Philadelphia, PA (US); The Trustees of the University of Pennsylvania, Philadelphia, PA (US)

(72) Inventors: Kristy B. Arbogast, Media, PA (US); Christina L. Master, Haverford, PA (US); Daniel Corwin, Philadelphia, PA (US); Matthew Grady, Philadelphia, PA (US); Catherine McDonald, Haverford, PA (US); Francesca Mandell, Washington, DC (US); Ian Barnett, Mount Laurel, NJ (US)

(73) Assignees: The Children's Hospital of Philadelphia, Philadelphia, PA (US); The Trustees of the University of Pennsylvania, Philadelphia, PA (US)

(21) Appl. No.: 18/822,614

(22) Filed: Sep. 3, 2024

Related U.S. Application Data
(60) Provisional application No. 63/539,410, filed on Sep. 20, 2023.

(51) Int. Cl. A61B 3/11; A61B 3/00; A61B 5/00

(52) U.S. Cl. CPC A61B 3/112; A61B 3/0008; A61B 5/4064; A61B 5/7275

(57) ABSTRACT

A portable pupillometer for evaluating one or more pupils of a subject includes an optical assembly to stimulate the one or more pupils. The optical assembly has a light source to deliver a stimulus to the one or more pupils and a detector to capture images of the one or more pupils during delivery of the stimulus. A control device is coupled to the optical assembly, which has a controller to perform a plurality of operations in accordance with instructions stored in a digital memory. The plurality of operations includes: obtaining pupillary light reflex data comprising a plurality of Pupillary Light Reflex metrics during stimulation of the one or more pupils; analyzing the pupillary light reflex data based on one or more characteristics of the subject; determining a probability of a concussion injury suffered by the subject; and outputting the probability to a user of the pupillometer.

### STATEMENT REGARDING FEDERALLY SPONSORED RESEARCH

This invention was made with government support under R01NS097549 awarded by the National Institute of Health. The government has certain rights in the invention.

### CROSS-REFERENCE TO RELATED APPLICATIONS

This application claims priority to U.S. patent application Ser. No. 63,539/410, entitled “PUPILLOMETER,” filed Sep. 20, 2023, the contents of which are incorporated herein by reference in their entirety.

### FIELD OF THE INVENTION

The subject matter disclosed herein relates to a portable pupillometer for evaluating one or more pupils of a subject.

### BACKGROUND OF THE INVENTION

Concussion (such as a sports-related concussion) is a common injury, in children and adolescents. One of the challenges for concussed youth is identifying those at risk for prolonged recovery. Most recover within 1 month of injury, however up to 30% experience persistent post-concussion symptoms (PPCS), defined as symptoms above baseline persisting beyond 28 days. However, early implementation of concussion-specific therapies can help expedite recovery when prescribed early in the course of injury, thereby making acute identification of those at highest risk for PPCS critically important.

Previous studies have identified individual risk factors associated with prolonged symptoms: (1) age; (2) sex; and (3) certain co-morbidities, such as prior concussion history and a history of migraine headaches. In addition, elements of commonly used clinical concussion batteries, such as the Sports Concussion Assessment Tool, 5th edition (SCAT-5), the King-Devick (K-D) test, symptom scales (e.g. the Post-Concussion Symptom Inventory (PCSI)), balance testing (e.g. modified balance error scoring system (mBESS)), and visio-vestibular testing deficits (e.g. visio-vestibular examination (VVE)), have been associated with prolonged symptom recovery in concussed youth. Further, the 5P rule is one example of a predictive model that combines risk factors to aid health care providers in making a judgment regarding a probability of a concussion injury.

The pupillary light reflex (PLR) represents an objective measure of the autonomic nervous system. Measured by a pupillometer, the PLR can express deficits as a result of a concussion. Improved pupillometers are desired, particularly improved pupillometers capable of combining various risk factors or stratifying risk to output a prediction regarding a probability of a concussion.

### SUMMARY OF THE INVENTION

A portable pupillometer for evaluating one or more pupils of a subject is disclosed. The portable pupillometer includes an optical assembly and a control device coupled to the optical assembly. The optical assembly is configured to stimulate the one or more pupils. The optical assembly includes a light source and a detector. The light source is configured to deliver a stimulus to the one or more pupils and the detector is configured to capture images of the one or more pupils during delivery of the stimulus. The control device has a controller configured to perform a plurality of operations in accordance with instructions stored in a digital memory. The plurality of operations includes: obtaining pupillary light reflex data during stimulation of the one or more pupils by the optical assembly, the pupillary light reflex data comprising a plurality of Pupillary Light Reflex (PLR) metrics; analyzing the pupillary light reflex data based on one or more characteristics of the subject; determining a probability of a concussion injury suffered by the subject; and outputting the probability to a user of the pupillometer.

### BRIEF DESCRIPTION OF THE FIGURES

The invention is best understood from the following detailed description when read in connection with the accompanying drawings, with like elements having the same reference numerals. When a plurality of similar elements are present, a single reference numeral may be assigned to the plurality of similar elements with a small letter designation referring to specific elements. When referring to the elements collectively or to a non-specific one or more of the elements, the small letter designation may be dropped. According to common practice, the various features of the drawings are not drawn to scale unless otherwise indicated. On the contrary, the dimensions of the various features may be expanded or reduced for clarity.

FIGS. 1A-1B depict schematic diagrams of an exemplary portable pupillometer, according to an aspect of the disclosure;

FIG. 2 is a flow chart of a predictive model performed by the pupillometer of FIG. 1, according to an aspect of the disclosure;

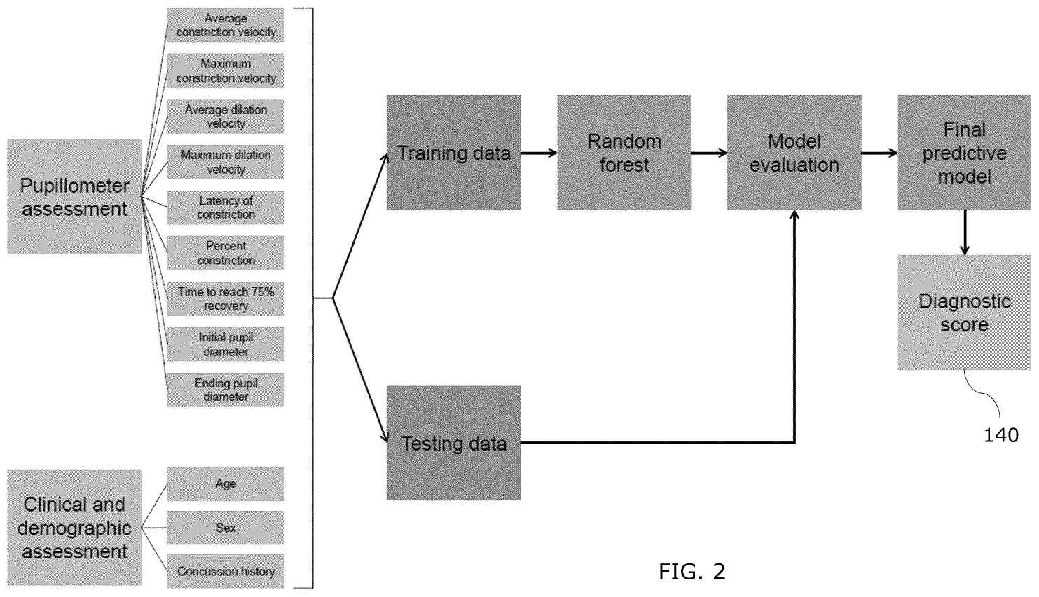

FIG. 3 depicts graphs showing a comparison of exemplary pupillary light reflex (PLR) metrics observed in concussed subjects compared with non-concussed or control subjects, according to assessments performed with the improved pupillometer of FIG. 1;

FIG. 4 depicts plots showing a distribution of PLR metrics observed in concussed subjects compared with non-concussed or control subjects, according to assessments performed with the improved pupillometer of FIG. 1;

FIG. 5 depicts plots showing a comparison of PLR metrics observed in subjects 7 days post-injury versus non-concussed or control subjects, according to assessments performed with the improved pupillometer of FIG. 1;

FIG. 6 depicts plots showing a comparison of female subjects to male subjects among concussed subjects and non-concussed or control subjects, according to assessments performed with the improved pupillometer of FIG. 1;

FIG. 7 depicts plots showing influence of exercise on PLR metrics in non-concussed or control subjects, according to assessments performed with the improved pupillometer of FIG. 1;

FIG. 8 depicts plots showing a comparison of concussed subjects and non-concussed or control subjects with and without history of prior concussion, according to assessments performed with the improved pupillometer of FIG. 1;

FIG. 9 depicts a graph showing a distribution of the PLR metrics among concussed subjects with non-concussed or control subjects, according to assessments performed with the improved pupillometer of FIG. 1; and

FIG. 10 depicts a graph showing results of an assessment of the predictive performance of the model of FIG. 2, according to assessments performed with the improved pupillometer of FIG. 1.

### DETAILED DESCRIPTION OF THE INVENTION

In the following detailed description, numerous specific details are set forth by way of examples in order to provide a thorough understanding of the relevant teachings. However, it should be apparent to those skilled in the art that the present teachings may be practiced without such details. In other instances, well known methods, procedures, components, and circuitry have been described at a relatively high-level, without detail, in order to avoid unnecessarily obscuring aspects of the present teachings.

Additionally, various forms and embodiments of the invention are illustrated in the figures. It will be appreciated that the combination and arrangement of some or all features of any of the embodiments with other embodiments is specifically contemplated herein. Accordingly, this detailed disclosure expressly includes the specific embodiments illustrated herein, combinations and sub-combinations of features of the illustrated embodiments, and variations of the illustrated embodiments.

Terms concerning attachments, coupling and the like, such as “coupled,” “mounted,” “connected” and “interconnected,” refer to a relationship wherein structures are secured or attached to one another either directly or indirectly through intervening structures, as well as both movable or rigid attachments or relationships, unless expressly described otherwise.

It will be understood that the terms and expressions used herein have the ordinary meaning as is accorded to such terms and expressions with respect to their corresponding respective areas of inquiry and study except where specific meanings have otherwise been set forth herein. The terms “comprises,” “comprising,” “includes,” “including,” or any other variation thereof, are intended to cover a non-exclusive inclusion, such that a process, method, article, or apparatus that comprises or includes a list of elements or steps does not include only those elements or steps but may include other elements or steps not expressly listed or inherent to such process, method, article, or apparatus. An element preceded by “a” or “an” does not, without further constraints, preclude the existence of additional identical or non-identical elements in the process, method, article, or apparatus that comprises the element.

Unless otherwise stated, any and all measurements, values, ratings, positions, magnitudes, sizes, and other specifications that are set forth in this specification, including in the claims that follow, are approximate, not exact. Such amounts are intended to have a reasonable range that is consistent with the functions to which they relate and with what is customary in the art to which they pertain, and taking into account manufacturing tolerances and typical variations in functional parameters. For example, unless expressly stated otherwise, a parameter value or the like may vary by as much as +10% from the stated amount.

Referring generally to the figures, the devices described herein enable improved evaluation of the characteristics of one or more pupils of a subject and improved output information based on said characteristics. The devices described herein generally include an optical assembly and a control device coupled to the optical assembly. In this way, a concussion injury may be evaluated by stratifying various risk factors according to a predictive model, such that a prediction regarding a concussion injury is outputted, thereby triggering medical personnel to behave/act in response to the prediction (e.g. assist medical personnel in making a health related judgment or assessment related to the subject and/or the concussion injury to the subject). As used herein and throughout the specification, users of the improved pupillometer is not limited to a specific medical personnel or health care professional, but may encompass other users (e.g. coaches, supervisors, trained or certified technicians, etc.) and in various situations not limited to a health care setting (e.g. at sports events, constructions sites, etc.) Still further, the term “subject” includes a patient, but is not limited to a specific patient and can encompass any and all types of patients, regardless of sex, age, and/or other physiological factors. Likewise, the term “concussion injury” (or “concussion” or “concussion event”) is not intended to be limited to a specific concussion or head impact/injury (of various degrees, kinds) or to specific symptom(s) related to head injuries, generally.

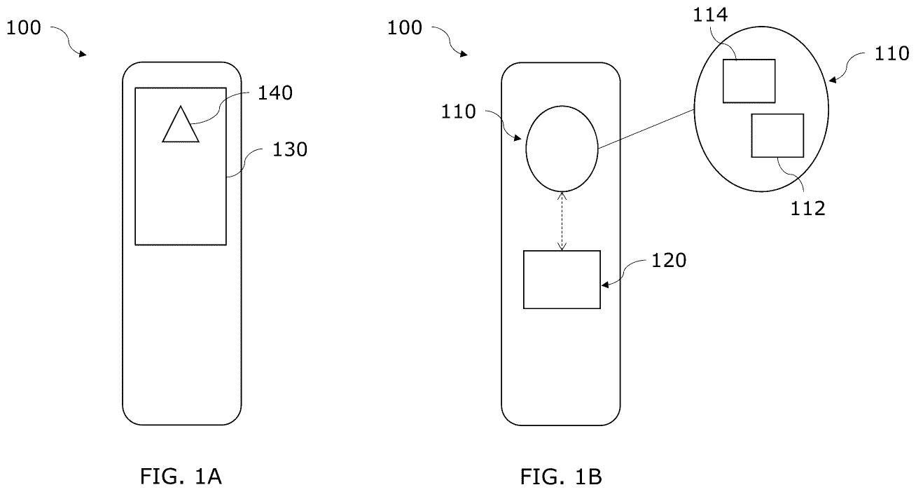

FIGS. 1A-1B are schematic diagrams of an exemplary portable pupillometer for evaluating one or more pupils of a subject, such as the pupillometer 100. In an exemplary embodiment, the portable pupillometer 100 includes an optical assembly 110 configured to stimulate the one or more pupils and a control device 120 coupled to the optical assembly 110. The optical assembly 110 has a light source 112 (e.g. LED light source) configured to deliver a stimulus to the one or more pupils of the subject and a detector 114 (e.g. an infrared camera) configured to capture images of the one or more pupils during delivery of the stimulus.

The control device 120 includes a controller configured to perform a plurality of operations in accordance with instructions stored in a digital memory. The plurality of operations can include controlling operation of the optical assembly 110, e.g. to operate light source 112 and/or detector 114 in accordance with one or more stored algorithms. The plurality of operations can further include analyzing and/or processing data received from the optical assembly 110, e.g. to calculate physiological characteristics or responses of the subject.

In an example, the plurality of operations includes obtaining pupillary light reflex data or characteristics during stimulation (e.g. when the light source 112 delivers a stimulus, such as a light stimulus, to the one or more pupils) of the one or more pupils by the optical assembly 110. During delivery of a light stimulus to a pupil, one or more characteristics (e.g. size or pupil diameter, light reflex, etc.) of the pupil in response to the stimulus can be observed and detected. These characteristics can be processed and stored by control device 120, and if desired, output to the user.

In an exemplary embodiment, the one or more characteristics of the pupil that can be observed includes pupillary light reflex data. The pupillary light reflex data includes a plurality of Pupillary Light Reflex (PLR) metrics, which includes one or more of: (1) an average constriction velocity of the one or more pupils (ACV); (2) a maximum constriction velocity of the one or more pupils (MCV); (3) an average dilation velocity of the one or more pupils (ADV); (4) a maximum dilation velocity of the one or more pupils (MDV); (45) a latency of constriction of the one or more pupils (LAT); (6) a percent constriction of the one or more pupils (CON); (7) a time for redilation of the one or more pupils from a minimum diameter to 75% maximum diameter (T75); (8) an initial pupil diameter of the one or more pupils (PDI); and (9) an ending pupil diameter of the one or more pupils (PDE).

Additionally or optionally, the plurality of operations also include analyzing the pupillary light reflex data based on one or more characteristics of the subject, such as clinical and demographic measurements, which may be input into the pupillometer using one or more input devices (e.g., buttons, keys, or via wired or wireless transmission from a separate device). In one example, one or more characteristics of the subject includes the subject's age (e.g. between 12 to 18 years old), sex, concussion history, a length of time between assessment and the event causing the concussion injury to the subject (e.g. less than 24 hours), or a combination thereof. In this way, the plurality of operations includes determining a probability of a concussion injury suffered by the subject, such as based on the observed pupillary light reflex data, the one or more characteristics of the subject, and/or a combination thereof, and outputting said probability to a user of the pupillometer 100. In one example, the pupillometer 100 includes a display 130 configured to output the probability to the user. Additionally or optionally, the concussion injury comprises persistent post-concussion symptoms (PPCS), such that analysis of the pupillary light reflex data comprises stratifying risk of PPCS in the concussed subject, e.g. into one or more risk categories. Additionally or optionally, the concussion injury comprises a sport related concussion (SRC), such that analysis of the pupillary light reflex data comprises stratifying risk of SRC in the concussed subject.

To achieve this, in an exemplary embodiment, the controller performs the analysis of the pupillary light reflex data according to a random forest algorithm. In an exemplary embodiment, the controller is configured to combine the PLR metrics and the one or more characteristics of the subject according to a random forest algorithm to determine the probability of a concussion injury suffered by the subject. Said probability may be indicated by a diagnostic score 140 representing the probability that an injured patient is concussed (e.g. a diagnostic score between 0 and 100). A random forest algorithm is an ensemble learning method that trains a collection of uncorrelated decision trees. A random forest function fθ(x) is predetermined in terms of the data (x) and a set of tunable parameters (θ), which includes the number of decision trees (e.g. 500) and the number of variables tried at each split in the decision tree (e.g. 3). Additionally, to improve the predictive performance on subjects assessed within 24 hours of injury, for example, a normal kernel weighting technique in sampling data is employed to grow each tree. In one example, the weights (w) may be defined as:

$${w}_{i} = \{ \begin{matrix}c ∅ \frac{{t}_{i} - t}{σ} & if {y}_{i} = 1 \\ 1 & if {y}_{i} = 0\end{matrix}$$

where i denotes the observation, ti is the observed time since injury, t=0, σ=2, γi=1 for concussed subjects and γi=0 for non-concussed subjects, Φ is the standard normal density function, and c is a constant selected such that Σᵢ₌₁ⁿI{yᵢ=1}wᵢ=Σᵢ₌₁ⁿ I{yᵢ=0} where n is the number of subjects in the data. This constant ensures equal contribution of concussed and non-concussed subjects to model estimation.

In an exemplary embodiment, the pupillometer 100 is configured to determine the probability of the concussion injury, based on the PLR metrics and the one or more characteristics of the subject, in accordance with instructions assigning predictive value (e.g. as measured by how much accuracy the predictive model loses by excluding each variable as predictors of concussion) as follows (from higher value to lower value): (1) PDI, (2) the subject's age; (3) MCV; (4) ACV; (5) MDV; (6) CON; (7) PDE; (8) T75; (9) ADV; (10) LAT; (11) history of concussion, and (12) sex.

### EXAMPLES

The co-inventors assessed feasibility and functionality of the components of the devices disclosed herein, as well as verified any updates or improvements made. For example, assessment of updates or improvements to the predictive model performed by the improved pupillometer is detailed herein.

### Example 1: Utility of Pupillary Light Reflex Metrics as a Physiologic Biomarker for Adolescent Sport-Related Concussion

Visual and autonomic dysfunction occur following sport related concussion (SRC), a form of mild traumatic brain injury (mTBI), negatively affecting adolescents in academic and athletic pursuits. Convergence and accommodation deficits after concussion predict prolonged recovery, while exercise intolerance is another manifestation of autonomic dysfunction after SRC. The pupillary light reflex (PLR) is involved in both convergence and accommodation, driven by the parasympathetic system for constriction and the sympathetic system for dilation. It can be quantitatively measured via dynamic, infrared pupillometry (DIP) in a rapid, reproducible manner. Quantitative PLR metrics may provide insight into autonomically influenced visual dysfunction following SRC, making the PLR a promising objective physiologic biomarker of concussion.

Normative values for PLR metrics have been described, with adults demonstrating decreasing pupil size with increasing age. In children across the age span, latency and average constriction/dilation velocities are similar; however, adolescents, specifically boys aged 12 to 18 years, have larger maximum pupil diameters, slower maximum constriction velocities, and smaller percentage constriction compared with younger children aged 6 to 11 years. These neurodevelopmental influences on the PLR11 make results of adult studies following concussion difficult to translate to children.

In the subacute phase after mTBI in adults, 2 to 8 weeks after injury, longer latency, lower average constriction/dilation velocities, and longer time to 75% pupillary redilation (T75) were found compared with control individuals. Another cross-sectional study in symptomatic adult chronic mTBI (greater than 3 months following injury) also found slower responses, with smaller initial pupil diameters, lower maximum and average constriction/dilation velocities, and lower constriction amplitudes compared with control individuals. In other adult studies, conflicting data have been reported, with one study finding no differences acutely after concussion but noting changes 2 to 4 weeks after injury, while another study found differences acutely (<72 hours following injury) compared with control individuals. In SRC, subclinical decrements in percentage pupil constriction and maximum constriction/dilation velocity were associated with high acceleration head impacts. The utility of pupillary assessment in SRC remains unclear, especially in children. Thus, the objective of this study was to determine whether differences in quantitative PLR metrics could serve as an objective physiologic biomarker for adolescent SRC.

### Methods

### Study Design, Setting, and Participants

Athletes aged 12 to 18 years were prospectively enrolled as part of a prospective observational cohort study. Athletes and/or their parents/legal guardians provided written assent/written informed consent. Healthy control individuals (n=143) were recruited from a private suburban high school with pupillometry assessments prior to their sport seasons. Athletes with a diagnosis of SRC (n=110) were recruited from a concussion program as well as the high school. Athletes with concussion enrolled after injury and thus did not have preinjury pupillometry. Ten participants who enrolled as healthy control individuals subsequently sustained an SRC and, for the purposes of this analysis, were included only in the concussed cohort. The diagnosis of SRC was made by a trained sports medicine pediatrician according to the most recent Consensus Statement on Concussion in Sports. All athletes with concussion had PLR assessments completed within 28 days of injury. If the injured patient had multiple assessments, the first assessment was used in this analysis. Exclusion criteria for both cases and controls included a concussion within 1 month of injury or preinjury assessment, ongoing chronic postconcussion symptoms, eye trauma, any ocular or neurologic condition, or medication that could affect pupillary responses. Forty six control individuals received gift cards as compensation for participation in the second year of the study. The remaining healthy athletes with concussion were not compensated for their participation.

### Instrumentation

Pupillary dynamics were measured in response to a brief, step input, white light stimulus (154 milliseconds' duration; 180 microwatts' power) via pupillometer. The pupillometer captures dynamic responses 32 times per second, analyzing a continuous, 5-second, digital video of the pupillary response to light. Eight metrics are quantified by the device software: maximum pupil diameter (steady-state pupil size before the light stimulus); minimum pupil diameter (pupil size after maximum constriction in response to the light stimulus); percentage pupil constriction; latency (time to maximum constriction in response to the light stimulus); peak and average constriction velocity; average dilation velocity; and T75 (time for pupil redilation from minimum diameter to 75% maximum diameter). A ninth metric, peak dilation velocity, was calculated from automated slope-based measures obtained by the pupillometer (Microsoft Excel 2016; Microsoft Corporation).

### Procedures

Trained research staff conducted PLR assessments in an athletic training room or sports medicine office, with a room illumination of approximately 350 lux (moderate photopic viewing conditions) and were not blinded to athlete concussion status because pupillometry is an objective measure requiring no subjective interpretation of results. Athletes focused on a 3-m distance target with the nontested eye for ocular fixation and accommodation during the 5-second measurement period. Monocular measurements were repeated at least 3 times for each eye, alternating 1-minute time intervals to allow rapid visual light adaptation, to obtain 2 to 3 artifact-free responses per eye. The combined mean of each pupillometry metric was calculated for at least 2 assessments without artifacts, defined as blinks or eye movements occurring within the first 3 seconds of the response. Approximately 6% of data were removed from each cohort owing to artifacts. This simple objective criterion approach to artifact removal with minimal post processing was used to maximize the translational potential of this paradigm to future clinical settings, including the sideline. Only artifact-free responses were analyzed. Among control individuals, staff recorded whether the assessment was conducted before or within 60 minutes after practice with each control assessed under one condition only. No athletes with concussion exercised before assessment.

### Statistical Analyses

Distribution of demographic and clinical characteristics for athletes with concussion were compared with controls using X² statistics for categorical variables (sex, race/ethnicity, and history of prior concussion) and F tests for age. The means of the PLR metrics were compared among athletes with concussion and controls with 1-way analysis of variance using F tests. Multiplicity was accounted for by calculating Bonferroni corrections for the 9 PLR metrics; the 99.44% CIs were presented around the mean values and the mean differences between comparison groups. Additionally, receiver operating characteristic curves and area under the curve were calculated for each metric. The planned primary analysis was based on previously published work. In sensitivity analyses, the analysis was further stratified by comparing the subgroup of athletes with concussion within 7 days of injury. In exploratory analyses, PLR metrics were examined within athletes with concussion and controls by sex, by history of prior concussion, and between those who did and did not exercise before assessment. With Bonferroni correction, the level of significance (a) was 0.0056 (0.05/9). 2-sided tests of statistical significance were used. Analyses were conducted using SAS software, version 9.4 (SAS Institute Inc).

### Results

### Study Population

Of the 253 athletes enrolled, valid PLR assessments were obtained for 134 of 143 healthy control individuals (93.7%) and 98 of 110 athleteswith concussion (89.1%). Among those without a valid assessment, 7 were too symptomatic to continue (e.g., eye pain/irritation, light sensitivity, and eye fatigue; 2 control individuals and 5 with concussion), 4 could not keep eyes open (2 control individuals and 2 with concussion), and 10 had insufficient analyzable measurements owing to artifact (<2 valid measurements for at least 1 eye; 5 control individuals and 5 with concussion). Among participants with evaluable pupillometry measurements, athletes with concussion (n=98) and control individuals (n=134) did not differ with respect to sex or race/ethnicity. Compared with controls, athletes with concussion were slightly older (median age, 15.7 years vs 15.2 years) and more likely to have a history of prior concussion (50% vs 26%). Athletes with concussion had pupillometry assessments performed a median of 12 days following injury (interquartile range [IQR], 5-18) (Table 1).

TABLE 1
Demographic and Clinical Characteristics of the Study Cohort

| | No. (%) Athletes | | Healthy control | |
|---|---|---|---|---|
| | with Concussion | | participants | |
| Characteristic | (n = 98) | | (n = 134) | |
| Age, mean (STD), y | 15.7 | (1.54) | 15.3 | (1.61) |
| Sex, Female | 55 | (56) | 78 | (58) |
| Sex, Male | 43 | (44) | 56 | (42) |
| Race/ethnicity, Non-Hispanic White | 83 | (85) | 109 | (81) |
| Race/ethnicity, Non-Hispanic Black | 8 | (8) | 11 | (8) |
| Race/ethnicity, Other/unknown | 7 | (7) | 14 | (10) |
| History of prior concussion*, NO | 47 | (48) | 99 | (74) |
| History of prior concussion*, YES | 50 | (51) | 35 | (26) |

*one athlete with concussion did not have history of prior concussion documented

### PLR Metrics in Concussion

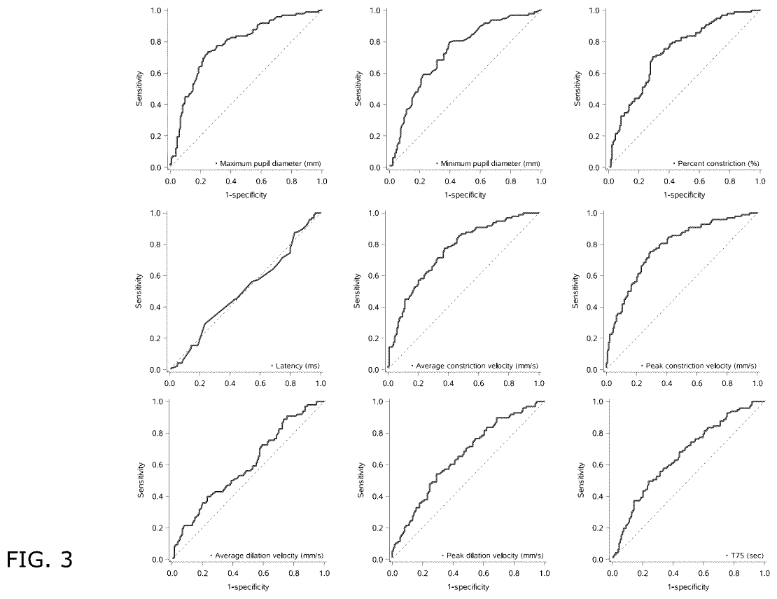

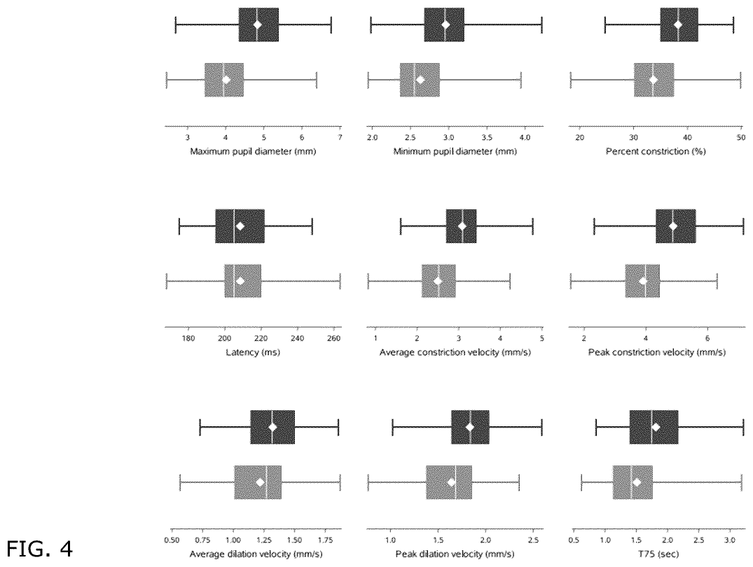

There were significant differences between athletes with concussion and controls for all PLR metrics except latency, after Bonferroni correction for multiple comparisons (Table 2). Athletes with concussion had larger maximum pupil diameter (4.83 mm vs 4.01 mm; difference, 0.82; 99.440/CI, 0.53-1.11), minimum pupil diameter (2.96 mm vs 2.63 mm; difference, 0.33; 99.40/CI, 0.18-0.48), and greater percentage constriction (38.230/vs 33.660%; difference, 4.57; 99.40/CI, 2.60-6.55). Additional enhanced PLR metrics (higher average constriction velocity, 3.08 mm/s vs 2.50 mm/s; difference, 0.58; 99.40/CI, 0.36-0.81), peak constriction velocity (4.88 mm/s vs 3.91 mm/s; difference, 0.97; 99.40/CI, 0.63-1.31), average dilation velocity (1.32 mm/s vs 1.22 mm/s, difference, 0.10; 99.40/CI, 0.00-0.20), peak dilation velocity (1.83 mm/s vs 1.64 mm/s; difference, 0.19; 99.4% CI, 0.07-0.32), and T75 (1.81 seconds vs 1.51 seconds; difference, 0.30; 99.4% CI, 0.10-0.51) were observed in athletes with concussion compared with control individuals. Receiver operating characteristic curves were plotted for each PLR metric, with maximum pupil diameter and peak constriction velocity achieving the greatest area under the curve (both=0.78) in distinguishing athletes with concussion from control individuals (FIG. 3, showing receiver operating characteristic curves for pupillary light reflex metrics in concussion). Anisocoria was not clinically observed in anyone in either cohort nor detected by pupillometry (mean [SD] pupillary diameter [MPD] among healthy control individuals, 4.08 mm right eye vs 3.94 [0.85] mm left eye; among patients with concussion, MPD [SD], 4.80 [0.83] mm right eye and 4.72 [0.76] mm in left eye; both nonsignificant). The median minimum and maximum pupillary diameters were also normally distributed (FIG. 4, showing boxplots of distribution of pupillary light reflex metrics in concussed athletes compared to healthy controls. Top boxplots (darker gray) represent concussed athletes and bottom boxplots (lighter gray) represent healthy controls with diamond as mean value, vertical white line as median value, box edges as 1st and 3rd quintile, whisker caps as minimum and maximum).

TABLE 2
Pupillary Light Reflex Metrics Distinguishing Healthy Control Participants
and Athletes With Concussion Within 28 Days Following Injury

| | Mean (99.44% CI) | | | |
|---|---|---|---|---|
| | Athletes with | Healthy control | | |
| | concussion | participants | | AUC* |
| Variable | (n = 98) | (n = 134) | Difference | (95% CI) |
| Pupil diameter, | 4.83 (4.62 | 4.01 (3.83 | 0.82 (0.53 | 0.78 (0.72 |
| mm, Maximum | to 5.05) | to 4.20) | to 1.11) | to 0.84) |
| Pupil diameter, | 2.96 (2.84 | 2.63 (2.53 | 0.33 (0.18 | 0.73 (0.67 |
| mm, Minimum | to 3.08) | to 2.73) | to 0.48) | to 0.80) |
| % Constriction | 38.23 (36.73 | 33.66 (32.38 | 4.57 (2.60 | 0.74 (0.67 |
| | to 39.74) | to 34.95) | to 6.55) | to 0.80) |
| Latency, ms | 208.40 (203.56 | 208.50 (204.36 | −0.09 (−6.46 | 0.50 (0.43 |
| | to 213.24) | to 212.63) | to 6.27) | to 0.58) |
| Constriction | 3.08 (2.91 | 2.50 (2.35 | 0.58 (0.36 | 0.76 (0.70 |
| velocity, mm/s, | to 3.25) | to 2.64) | to 0.81) | to 0.82) |
| Average | | | | |
| Constriction | 4.88 (4.62 | 3.91 (3.69 | 0.97 (0.63 | 0.78 (0.72 |
| velocity, mm/s, | to 5.14) | to 4.13) | to 1.31) | to 0.84) |
| Peak | | | | |
| Dilation velocity, | 1.32 (1.24 | 1.22 (1.15 | 0.10 (0.00 | 0.60 (0.52 |
| mm/s, Average | to 1.40) | to 1.28) | to 0.20) | to 0.67) |
| Constriction | 1.83 (1.74 | 1.64 (1.56 | 0.19 (0.07 | 0.66 (0.59 |
| velocity, mm/s, | to 1.93) | to 1.72) | to 0.32) | to 0.73) |
| Peak | | | | |
| T75*, s | 1.81 (1.66 | 1.51 (1.38 | 0.30 (0.10 | 0.65 (0.58 |
| | to 1.97) | to 1.64) | to 0.51) | to 0.72) |

*AUC: area under the curve; T75, time to 75% pupillary redilation.

### Sensitivity Analyses of Subgroups

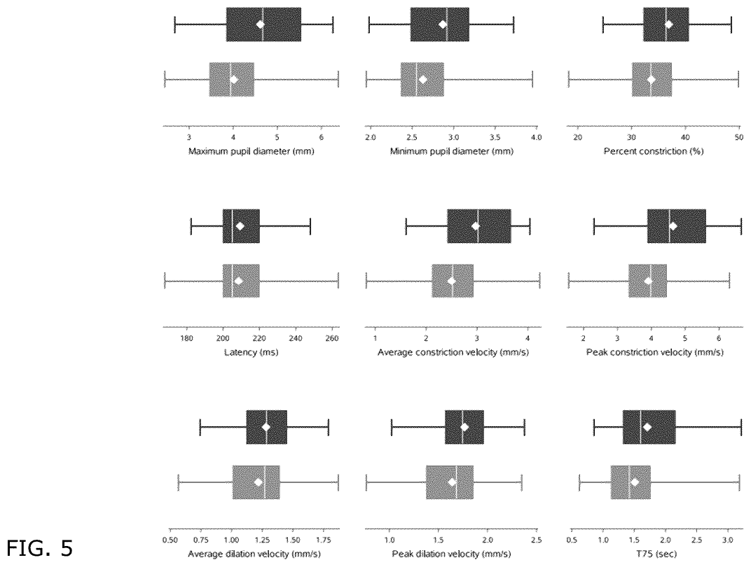

A subgroup of athletes with acute concussion assessed within 7 days following injury (n=35) were compared with control participants. After Bonferroni correction, differences between control participants and athletes with concussion within 7 days following injury were no longer significant for average dilation velocity and T75 but continued to be significant for the remaining 7 parameters (Table 3 and FIG. 5, showing pupillary light reflex metrics distinguish healthy controls and acutely concussed athletes≤7 days post-injury. Top=cases, bottom=controls). Next, no sex differences were found in control participants for any metric.

TABLE 3
Pupillary Light Reflex Metrics Distinguish Healthy Controls
and Acutely Concussed Athletes ≤7 days Post-Injury

| | Concussed | | Healthy | | | | |
|---|---|---|---|---|---|---|---|
| | athletes | | controls | | Difference | | |
| | (n = 35) | | (n = 134) | | | | Bonferroni |
| | Mean | | Mean | | Mean | | adjusted |
| Variable | (99.44% CI) | | (99.44% CI) | | (99.44% CI) | | P value |
| Maximum pupil | 4.61 | (4.23, 5.00) | 4.01 | (3.82, 4.21) | 0.60 | (0.17, 1.03) | .001 |
| diameter (mm) | | | | | | | |
| Minimum pupil | 2.87 | (2.67, 3.07) | 2.63 | (2.53, 2.73) | 0.24 | (0.01, 0.46) | .03 |
| diameter (mm) | | | | | | | |
| Percent | 36.91 | (34.21, 39.61) | 33.66 | (32.28, 35.04) | 3.25 | (0.21, 6.28) | .03 |
| constriction | | | | | | | |
| (%) | | | | | | | |
| Latency (ms) | 209.29 | (200.94, 217.64) | 208.50 | (204.23, 212.76) | 0.79 | (−8.58, 10.17) | 1 |
| Average | 2.97 | (2.67, 3.27) | 2.50 | (2.34, 2.65) | 0.47 | (0.13, 0.81) | .001 |
| constriction | | | | | | | |
| velocity | | | | | | | |
| (mm/s) | | | | | | | |
| Peak | 4.64 | (4.18, 5.10) | 3.91 | (3.68, 4.14) | 0.73 | (0.22, 1.24) | <.001 |
| constriction | | | | | | | |
| velocity | | | | | | | |
| (mm/s) | | | | | | | |
| Average | 1.28 | (1.15, 1.41) | 1.22 | (1.15, 1.28) | 0.06 | (−0.08, 0.21) | 1 |
| dilation | | | | | | | |
| velocity | | | | | | | |
| (mm/s) | | | | | | | |
| Peak | 1.77 | (1.61, 1.93) | 1.64 | (1.56, 1.72) | 0.13 | (−0.05, 0.31) | .44 |
| dilation | | | | | | | |
| velocity | | | | | | | |
| (mm/s) | | | | | | | |
| T75 (s) | 1.71 | (1.44, 1.97)* | 1.51 | (1.38, 1.64) | 0.20 | (−0.10, 0.49) | .53 |

*1 concussed athlete did not have at least valid measures for the T75 metric

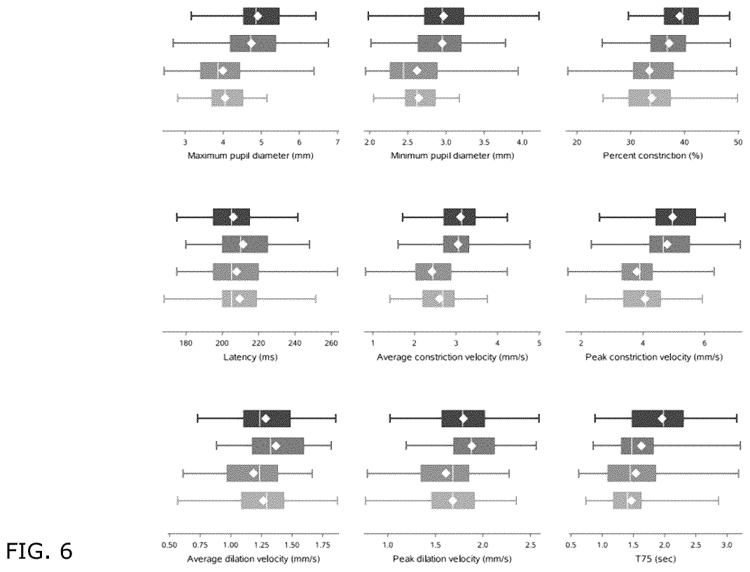

After Bonferroni correction, differences were observed, with girls with concussion exhibiting longer T75 (1.96 seconds vs 1.63 seconds; difference, 0.33; 99.40/CI, 0.02-0.65) (Table 4 and FIG. 6, showing a comparison of females to males among concussed athletes and healthy controls. Top=female cases, 2nd from the top=male cases, 3rd from the top=female controls, bottom=male controls).

TABLE 4
Sex-Differences in T75 Among Concussed Athletes, But Not Healthy Controls

| | Female Concussed | | Male Concussed | | | | |
|---|---|---|---|---|---|---|---|
| | Athletes | | Athletes | | Difference | | |
| | (n = 55) | | (n = 43) | | | | Bonferroni |
| | Mean | | Mean | | Mean | | adjusted |
| Variable | (99.44% CI) | | (99.44% CI) | | (99.44% CI) | | P value |
| Maximum pupil | 4.91 | (4.61, 5.20) | 4.74 | (4.41, 5.07) | 0.17 | (−0.27, 0.61) | 1 |
| diameter (mm) | | | | | | | |
| Minimum pupil | 2.97 | (2.81, 3.13) | 2.95 | (2.77, 3.13) | 0.02 | (−0.22, 0.25) | 1 |
| diameter (mm) | | | | | | | |
| Percent | 39.09 | (37.28, 40.90) | 37.14 | (35.09, 39.18) | 1.96 | (−0.78, 4.69) | .4 |
| constriction | | | | | | | |
| (%) | | | | | | | |
| Latency (ms) | 206.03 | (199.90, 212.17) | 211.43 | (204.49, 218.37) | −5.40 | (−14.65, 3.86) | .91 |
| Average | 3.11 | (2.89, 3.33) | 3.05 | (2.80, 3.29) | 0.06 | (−0.27, 0.39) | 1 |
| constriction | | | | | | | |
| velocity | | | | | | | |
| (mm/s) | | | | | | | |
| Peak | 4.96 | (4.62, 5.30) | 4.78 | (4.39, 5.17) | 0.18 | (−0.34, 0.70) | 1 |
| constriction | | | | | | | |
| velocity | | | | | | | |
| (mm/s) | | | | | | | |
| Average | 1.28 | (1.18, 1.38) | 1.37 | (1.25, 1.48) | −0.08 | (−0.24, 0.07) | 1 |
| dilation | | | | | | | |
| velocity | | | | | | | |
| (mm/s) | | | | | | | |
| Peak dilation | 1.80 | (1.67, 1.92) | 1.88 | (1.74, 2.03) | −0.09 | (−0.28, 0.10) | 1 |
| velocity | | | | | | | |
| (mm/s) | | | | | | | |
| T75 (s) | 1.96 | (1.75, 2.17)* | 1.63 | (1.39, 1.86) | 0.33 | (0.02, 0.65) | .03 |
| | Female Healthy | | Male Healthy | | | | |
| | Controls | | Controls | | Difference | | |
| | (n = 78) | | (n = 56) | | | | Bonferroni |
| | Mean | | Mean | | Mean | | adjusted |
| | (99.44% CI) | | (99.44% CI) | | (99.44% CI) | | P value |
| Maximum pupil | 3.99 | (3.74, 4.24) | 4.05 | (3.76, 4.34) | −0.06 | (−0.44, 0.33) | 1 |
| diameter (mm) | | | | | | | |
| Minimum pupil | 2.62 | (2.49, 2.75) | 2.64 | (2.49, 2.80) | −0.02 | (−0.23, 0.18) | 1 |
| diameter (mm) | | | | | | | |
| Percent | 33.47 | (31.66, 35.28) | 33.93 | (31.79, 36.07) | −0.46 | (−3.27, 2.34) | 1 |
| constriction | | | | | | | |
| (%) | | | | | | | |
| Latency (ms) | 207.79 | (202.11, 213.48) | 209.48 | (202.77, 216.19) | −1.68 | (−10.48, 7.11) | 1 |
| Average | 2.42 | (2.23, 2.62) | 2.60 | (2.37, 2.84) | −0.18 | (−0.49, 0.12) | .88 |
| constriction | | | | | | | |
| velocity | | | | | | | |
| (mm/s) | | | | | | | |
| Peak | 3.80 | (3.50, 4.09) | 4.06 | (3.72, 4.41) | −0.26 | (−0.72, 0.19) | .93 |
| constriction | | | | | | | |
| velocity | | | | | | | |
| (mm/s) | | | | | | | |
| Average | 1.18 | (1.10, 1.27) | 1.26 | (1.16, 1.37) | −0.08 | (−0.21, 0.05) | .87 |
| dilation | | | | | | | |
| velocity | | | | | | | |
| (mm/s) | | | | | | | |
| Peak dilation | 1.61 | (1.50, 1.72) | 1.68 | (1.55, 1.81) | −0.07 | (−0.24, 0.10) | 1 |
| velocity | | | | | | | |
| (mm/s) | | | | | | | |
| T75 (s) | 1.54 | (1.37, 1.71) | 1.47 | (1.26, 1.67) | 0.07 | (−0.19, 0.34) | 1 |

*1 concussed athlete did not have at least 2 valid measures for the T75 metric

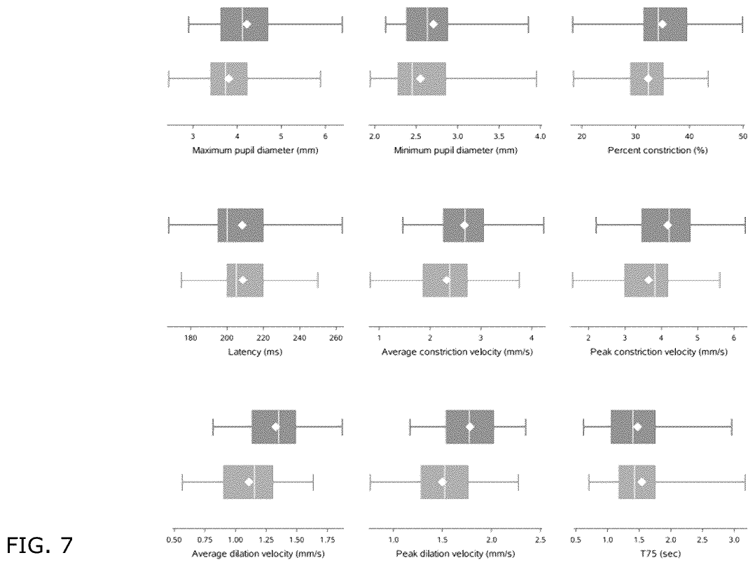

A subgroup of control participants assessed within 60 minutes after practice manifested smaller maximum pupil diameters, smaller maximum pupil size (3.81 mm vs 4.22 mm; difference, −0.41; 99.4%/CI, −0.77 to 0.05), lower average constriction velocity (2.32 mm/s vs 2.67 mm/s; difference, −0.35; 99.4%/CI, −0.64 to −0.06), peak constriction velocity (3.65 mm/s vs 4.17 mm/s; difference, −0.52; 99.4% CI, −0.96 to −0.09), average dilation velocity (1.11 mm/s vs 1.33 mm/s; difference, 99.4CI, −0.22; −0.34 to −0.10), and peak dilation velocity (1.50 mm/s vs 1.78 mm/s; difference, −0.28; 99.4CI, −0.44 to −0.13) compared with control individuals who did not exercise before assessment (Table 5 and FIG. 7, showing influence of exercise on pupillary light reflex metrics in healthy controls. Top=Healthy controls before exercise, bottom=Healthy controls after exercise). Pupillary light reflex metrics were similar between those with and without a history of prior concussion among control participants.

TABLE 5
Influence of Exercise on Pupillary Light Reflex Metrics in Healthy Controls

| | Female Healthy | | Male Healthy | | | | |
|---|---|---|---|---|---|---|---|
| | Controls | | Controls | | Difference | | |
| | (n = 78) | | (n = 56) | | | | Bonferroni |
| | Mean | | Mean | | Mean | | adjusted |
| Variable | (99.44% CI) | | (99.44% CI) | | (99.44% CI) | | P value |
| Maximum pupil | 4.22 | (3.96, 4.48) | 3.81 | (3.55, 4.07) | −0.41 | (−0.77, −0.05) | .02 |
| diameter (mm) | | | | | | | |
| Minimum pupil | 2.71 | (2.57, 2.85) | 2.55 | (2.41, 2.69) | −0.16 | (−0.36, 0.04) | .24 |
| diameter (mm) | | | | | | | |
| Percent | 34.98 | (33.08, 36.88) | 32.34 | (30.44, 34.24) | 2.64 | (−5.33, 0.05) | −.06 |
| constriction | | | | | | | |
| (%) | | | | | | | |
| Latency (ms) | 208.32 | (202.18, 214.46) | 208.67 | (202.53, 214.82) | 0.36 | (−8.33, 9.04) | 1 |
| Average | 2.67 | (2.47, 2.88) | 2.32 | (2.12, 2.53) | −0.35 | (−0.64, −0.06) | .009 |
| constriction | | | | | | | |
| velocity | | | | | | | |
| (mm/s) | | | | | | | |
| Peak | 4.17 | (3.86, 4.48) | 3.65 | (3.34, 3.95) | −0.52 | (−0.96, −0.09) | .008 |
| constriction | | | | | | | |
| velocity | | | | | | | |
| (mm/s) | | | | | | | |
| Average | 1.33 | (1.24, 1.41) | 1.11 | (1.02, 1.19) | −0.22 | (−0.34, −0.10) | <.001 |
| dilation | | | | | | | |
| velocity | | | | | | | |
| (mm/s) | | | | | | | |
| Peak dilation | 1.78 | (1.67, 1.89) | 1.50 | (1.39, 1.61) | −0.28 | (−0.44, −0.13) | <.001 |
| velocity | | | | | | | |
| (mm/s) | | | | | | | |
| T75 (s) | 1.47 | (1.29, 1.66) | 1.54 | (1.36, 1.73) | 0.07 | (−0.19, 0.33) | 1 |

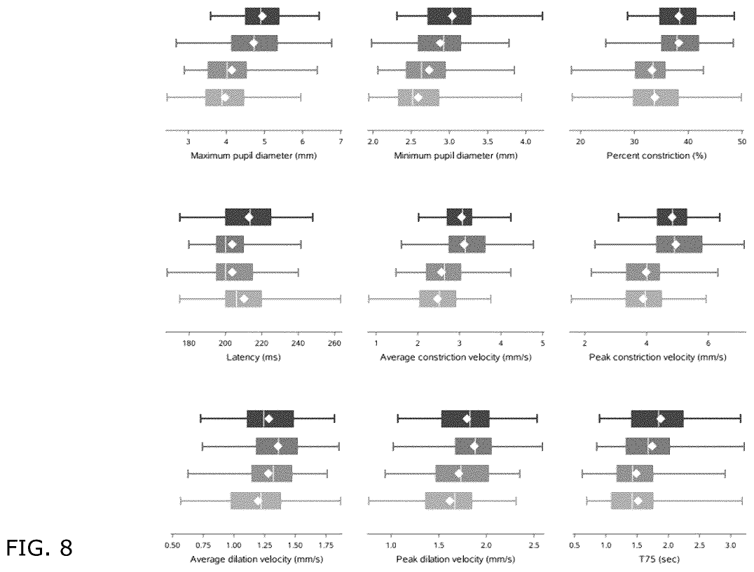

In athletes with concussion, those with a history of concussion had longer latency after Bonferroni correction (212.9 milliseconds vs 203.7 milliseconds; difference, 9.21; 99.4%/CI, 0.28-18.14) (Table 6 and FIG. 8, showing a comparison of concussed athletes and healthy controls with and without history of prior concussion. Top=cases with prior concussion, 2nd from the top=cases without prior concussion, 3rd from the top=controls with prior concussion, bottom=controls without prior concussion).

TABLE 6
Comparison of concussed athletes and uninjured controls with and without history of prior concussion

| | |
|---|---|
| Concussed | Concussed |
| athletes with | athletes without |
| prior history of | prior history of |

| | | |
|---|---|---|
| concussion | concussion | Difference |

| | (n = 50) | | (n = 48) | | | | Bonferroni |
|---|---|---|---|---|---|---|---|
| | Mean | | Mean | | Mean | | adjusted |
| Variable | (99.44% CI) | | (99.44% CI) | | (99.44% CI) | | P value |
| Maximum pupil | 4.94 | (4.64, 5.25) | 4.72 | (4.41, 5.03) | 0.22 | (−0.21, 0.66) | 1 |
| diameter (mm) | | | | | | | |
| Minimum pupil | 3.04 | (2.87, 3.20) | 2.88 | (2.71, 3.05) | 0.16 | (−0.07, 0.39) | .52 |
| diameter (mm) | | | | | | | |
| Percent | 38.23 | (36.30, 40.17) | 38.23 | (36.26, 40.21) | 0.00 | (−2.77, 2.77) | 1 |
| constriction | | | | | | | |
| (%) | | | | | | | |
| Latency (ms) | 212.91 | (206.66, 219.16) | 203.70 | (197.32, 210.08) | 9.21 | (0.28, 18.14) | .04 |
| Average | 3.05 | (2.82, 3.28) | 3.11 | (2.88, 3.35) | −0.06 | (−0.39, 0.27) | 1 |
| constriction | | | | | | | |
| velocity | | | | | | | |
| (mm/s) | | | | | | | |
| Peak | 4.82 | (4.46, 5.18) | 4.94 | (4.57, 5.31) | −0.12 | (−0.63, 0.40) | 1 |
| constriction | | | | | | | |
| velocity | | | | | | | |
| (mm/s) | | | | | | | |
| Average | 1.28 | (1.18, 1.39) | 1.36 | (1.25, 1.47) | −0.07 | (−0.22, 0.08) | 1 |
| dilation | | | | | | | |
| velocity | | | | | | | |
| (mm/s) | | | | | | | |
| Peak dilation | 1.80 | (1.66, 1.93) | 1.87 | (1.74, 2.01) | −0.08 | (−0.27, 0.11) | 1 |
| velocity | | | | | | | |
| (mm/s) | | | | | | | |
| T75 (s) | 1.88 | (1.65, 2.11) | 1.74 | (1.51, 1.98)* | 0.14 | (−0.19, 0.46) | 1 |

| | Healthy controls | | Healthy controls | | | | |
|---|---|---|---|---|---|---|---|
| | with prior | | without prior | | | | |
| | history of | | history of | | | | |
| | concussion | | concussion | | Difference | | |
| | (n = 35) | | (n = 99) | | | | Bonferroni |
| | Mean | | Mean | | Mean | | adjusted |
| | (99.44% CI) | | (99.44% CI) | | (99.44% CI) | | P value |
| Maximum pupil | 4.14 | (3.78, 4.51) | 3.97 | (3.75, 4.19) | 0.18 | (−0.25, 0.60) | 1 |
| diameter (mm) | | | | | | | |
| Minimum pupil | 2.74 | (2.54, 2.93) | 2.59 | (2.48, 2.71) | 0.14 | (−0.08, 0.37) | .7 |
| diameter (mm) | | | | | | | |
| Percent | 33.25 | (30.55, 35.96) | 33.80 | (32.20, 35.41) | −0.55 | (−3.69, 2.60) | 1 |
| constriction | | | | | | | |
| (%) | | | | | | | |
| Latency (ms) | 203.74 | (195.35, 212.13) | 210.18 | (205.19, 215.17) | −6.44 | (−16.20, 3.32) | .59 |
| Average | 2.57 | (2.27, 2.87) | 2.47 | (2.30, 2.65) | 0.10 | (−0.25, 0.44) | 1 |
| constriction | | | | | | | |
| velocity | | | | | | | |
| (mm/s) | | | | | | | |
| Peak | 3.98 | (3.54, 4.42) | 3.88 | (3.62, 4.15) | 0.10 | (−0.42, 0.61) | 1 |
| constriction | | | | | | | |
| velocity | | | | | | | |
| (mm/s) | | | | | | | |
| Average | 1.28 | (1.15, 1.41) | 1.20 | (1.12, 1.27) | 0.08 | (−0.07, 0.23) | 1 |
| dilation | | | | | | | |
| velocity | | | | | | | |
| (mm/s) | | | | | | | |
| Peak dilation | 1.71 | (1.55, 1.87) | 1.62 | (1.52, 1.71) | 0.09 | (−0.10,0.29) | 1 |
| velocity | | | | | | | |
| (mm/s) | | | | | | | |
| T75 (s) | 1.49 | (1.23, 1.74) | 1.52 | (1.36, 1.67) | −0.03 | (−0.33, 0.27) | 1 |

*1 concussed athlete did not have 3 valid measures for the T75 metric

### Analysis

This example demonstrates that quantitative PLR metrics obtained via DIP differentiate concussed adolescent athletes from healthy control participants. Enhancement of PLR metrics characterizes acute concussion, with larger pupil sizes and increased average and peak constriction/dilation velocities. The association of concussion with PLR metrics appears robust, with significant differences between athletes with concussion and control participants in all metrics except for latency. The results presented here may be clinically relevant because these metrics are easily obtained via automated dynamic infrared pupillometry, and the measurable objective differences discriminate well between adolescents with and without concussion, indicating potential future utility in the diagnosis of concussion in the sports setting.

### Enhancement of PLR Metrics in Concussion

The enhancement of PLR metrics reflects relative sympathetic predominance after concussion, which also affects exercise intolerance after concussion. Pupillary light reflex enhancement has been described in infants at risk for autism, attributed to possible cholinergic system disruption during infant neurodevelopment resulting in an excitatory-inhibitory autonomic imbalance. In concussion, an analogous, traumatically acquired, autonomic dysfunction is described, with excessive sympathetic tone. The results support this hypothesis with findings of larger maximum pupillary diameters, permitting more light to enter the eye, thus influencing the downstream dynamics of both constriction and dilation as evidenced by higher peak and average constriction/dilation velocities and greater percentage constriction in adolescent athletes with concussion. Of interest, PLR latency did not differ between athletes with concussion and control individuals, which might have been expected in concussion. However, the lack of slowing may be owing to the high-intensity light stimulus producing a response saturation effect, such that differences in latency might only be detected at lower luminosity.

### Sex-Based Differences in the PLR in Concussion

The results demonstrate prolongation of T75 in girls with concussion, lending support to previously described sex-based differences following concussion, reflecting differential adverse effects of trauma on the sympathetic system. There are no previously reported sex differences in T75 in control participants, in either adults or children, which is confirmed by the study, making the sex differences in T75 in girls with concussion of particular interest.

### Age-Related Differences in the PLR in Concussion

The results advance the quest for an objective physiologic biomarker for concussion, representing the report of PLR metrics distinguishing adolescent SRC from healthy control athletes. In contrast, adult studies of blast mTBI found smaller pupillary sizes and slower responses compared with healthy control participants. These differences may be neurodevelopmental in nature. First, age-related differences in the PLR exist. Within the healthy pediatric population, older adolescents aged 12 to 18 years have larger pupil diameters, slower peak constriction velocities, and smaller percentage constriction than younger children aged 6 to 11 years. These differences in the PLR in childhood likely influence findings after concussion. Second, differences in recovery trajectories for concussion in adolescents vs adults have been previously demonstrated. Cerebral blood flow changes following concussion take longer to recover in children compared with adults; recovery of the PLR may also be affected by developmental factors affecting the autonomic nervous system. Lastly, the timing of PLR assessments following injury may account for some of these differences with parameters changing over time after concussion. Our study assessed adolescents with concussion within 28 days of injury (acute to subacute time frame), whereas adult studies assessed symptomatic patients anywhere from 2 weeks to more than 3 months following injury (subacute to chronic time frame). Temporal changes in the PLR over the course of recovery may also account for observed differences.

### Association of Exercise With the PLR

The study also found that exercise had an effect on PLR metrics in control individuals. Those who exercised before pupillary assessment had smaller maximum and minimum pupil sizes with lower peak and average constriction/dilation velocities compared with control individuals who did not exercise before assessment. Pupillary light reflex metrics in the postexercise group were similar to those described in an adult cohort with mTBI in the chronic time frame, indicating that the physiology of the PLR in symptomatic chronic mTBI may be similar to physical fatigue.

Prospective work based on this study include efforts to examine multivariable modeling, including history of prior concussion and also the effect of using lower luminosity stimuli to determine whether differences in latency might be detected under these conditions. Additional studies could further explore PLR metrics in girls and after exercise, as well as longitudinally after concussion, to better understand PLR metrics as a potential objective physiologic biomarker for concussion and recovery. Further studies to confirm these findings beyond a single site, with attention to understanding the influence of lower luminosity and exercise, are warranted to determine whether PLR metrics have the potential to serve as quantitative physiologic biomarkers for adolescent SRC.

These results suggest that PLR metrics can serve as robust objective physiologic biomarkers for adolescent SRC, with athletes with concussion manifesting PLR enhancement. Longer pupillary recovery T75 times were noted in girls with concussion, indicating a potential biologic basis for sex differences in concussion. In control participants, slower PLR responses after exercise indicate a fatigue effect.

### Example 2: Modeling Approach

Quantitative pupillary light reflex (PLR) metrics can assess visual dysfunction following concussion, and therefore can be used to discriminate concussed from non-concussed patients.

The inventive pupillometer employs a predictive model that incorporates nine PLR metrics as well as sex, age, and concussion history to predict the probability that an injured patient is concussed. A random forest, a non-parametric ensemble learning method, is trained on subjects assessed between 0 and 28 days post-injury and sample weighting is employed to improve predictive performance on subjects assessed within 1 day of injury. Model performance is assessed on validation data using multiple classification metrics. Ultimately, this produces a model that uses the set of PLR metrics along with age, sex, and concussion history to predict a diagnostic score (such as from 0 to 100) that represents the probability that an injured patient is concussed. The model is trained to maximize discrimination between non-concussed and concussed subjects assessed within 1 day of injury.

### Study Data

TABLE 9
Demographic and clinical characteristics of the study cohort

| | |
|---|---|
| Concussed | Non-Concussed |
| (n = 257) | (n = 355) |

| | | | | |
|---|---|---|---|---|
| Mean age (sd) | 15.3 | (2.8) | 17.3 | (2.9) |
| Female sex (n, %) | 139 | (54.1%) | 124 | (34.9%) |
| History of prior | 97 | (37.7%) | 97 | (27.3%) |
| concussion (n, %) | | | | |

| | | | |
|---|---|---|---|
| Median days from injury | 6.0 | (14) | NA |
| to 1st visit (IQR) | | | |

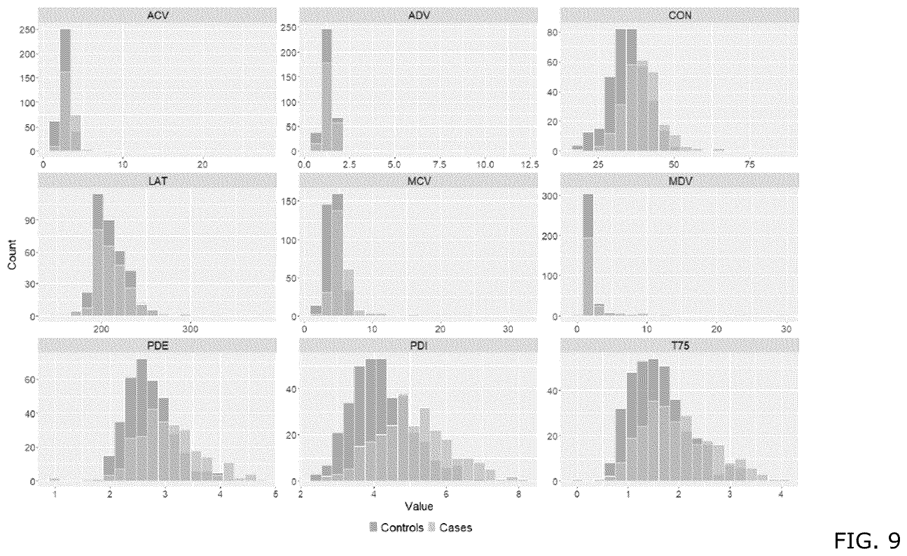

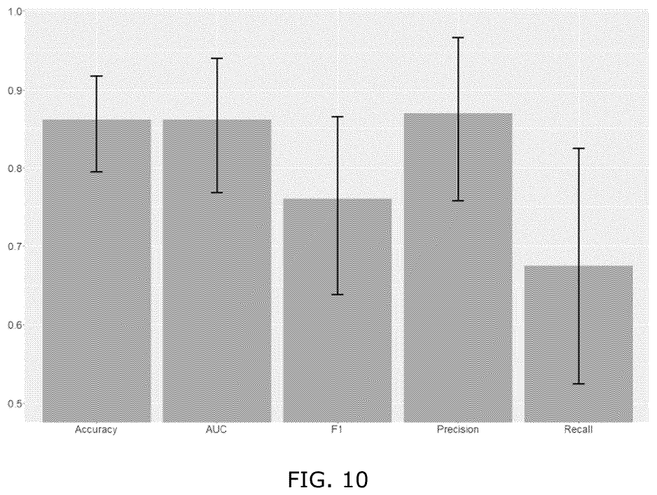

FIG. 9 shows distribution of nine PLR metrics by case status. Table 10 and FIG. 10 show classification metrics of the weighted random forest calculated on validation data, with 95% confidence intervals calculated from 1000 bootstrap samples.

TABLE 10
Classification metrics of the weighted random
forest calculated on validation data.

| Metric | Estimate | 95% CI |
|---|---|---|
| Accuracy | 0.861 | 0.795-0.918 |
| AUC | 0.861 | 0.769-0.941 |
| F1 | 0.761 | 0.638-0.865 |
| Precision | 0.871 | 0.759-0.967 |
| Recall | 0.675 | 0.525-0.825 |

While the foregoing has described what are considered to be the best mode and other examples, it is understood that various modifications may be made therein and that the subject matter disclosed herein may be implemented in various forms and examples, and that they may be applied in numerous applications, only some of which have been described herein. It is intended by the following claims to claim any and all modifications and variations that fall within the true scope of the present concepts.

What is claimed is:

1. A portable pupillometer for evaluating one or more pupils of a subject, the pupillometer comprising:
an optical assembly configured to stimulate the one or more pupils, the optical assembly comprising:
a light source configured to deliver a stimulus to the one or more pupils; and
a detector configured to capture images of the one or more pupils during delivery of the stimulus; and
a control device coupled to the optical assembly, the control device comprising a controller configured to perform a plurality of operations in accordance with instructions stored in a digital memory, wherein the plurality of operations includes:
obtaining pupillary light reflex data during stimulation of the one or more pupils by the optical assembly, the pupillary light reflex data comprising a plurality of Pupillary Light Reflex (PLR) metrics;
analyzing the pupillary light reflex data based on one or more characteristics of the subject;
determining a probability of a concussion injury suffered by the subject; and
outputting the probability to a user of the pupillometer.

2. The pupillometer of claim 1, wherein the plurality of PLR metrics includes an average constriction velocity of the one or more pupils (ACV).

3. The pupillometer of claim 1, wherein the plurality of PLR metrics includes a maximum constriction velocity of the one or more pupils (MCV).

4. The pupillometer of claim 1, wherein the plurality of PLR metrics includes an average dilation velocity of the one or more pupils (ADV).

5. The pupillometer of claim 1, wherein the plurality of PLR metrics includes a maximum dilation velocity of the one or more pupils (MDV).

6. The pupillometer of claim 1, wherein the plurality of PLR metrics includes a latency of constriction of the one or more pupils (LAT).

7. The pupillometer of claim 1, wherein the plurality of PLR metrics includes a percent constriction of the one or more pupils (CON).

8. The pupillometer of claim 1, wherein the plurality of PLR metrics includes a time for redilation of the one or more pupils from a minimum diameter to 75% maximum diameter (T75).

9. The pupillometer of claim 1, wherein the plurality of PLR metrics includes an initial pupil diameter of the one or more pupils (PDI).

10. The pupillometer of claim 1, wherein the plurality of PLR metrics includes an ending pupil diameter of the one or more pupils (PDE).

11. The pupillometer of claim 1, wherein the one or more characteristics of the subject comprise the subject's age, sex, concussion history, or combination thereof.

12. The pupillometer of claim 1, wherein the one or more characteristics of the subject include a length of time from an event causing the concussion injury to the subject.

13. The pupillometer of claim 12, wherein the length of time is less than twenty four hours from the event causing the concussion injury to the head of the subject.

14. The pupillometer of claim 1, wherein the controller performs the analysis of the pupillary light reflex data according to a random forest algorithm.

15. The pupillometer of claim 1, wherein analysis of the pupillary light reflex data comprises stratifying risk of persistent post-concussion symptoms in the concussed subject.

16. The pupillometer of claim 1, further comprising a display configured to output the probability to the user.

17. The pupillometer of claim 1, wherein the one or more characteristics of the subject comprise the subject's age and the plurality of PLR metrics comprise an initial pupil diameter of the one or more pupils.

18. The pupillometer of claim 1, wherein the plurality of PLR metrics includes ACV, MCV, ADV, MDV, LAT, CON, T75, PDI, PDE, or a combination thereof.

19. The pupillometer of claim 18, wherein the controller assigns a decreasing predictive value to the plurality of PLR metrics and the one or more characteristics of the subject, according to the list of: PDI, the subject's age; (3) MCV; (4) ACV; (5) MDV; (6) CON; (7) PDE; (8) T75; (9) ADV; (10) LAT; (11) the subject's concussion history, and (12) the subject's sex.
